(19) United States
(12) Patent Application Publication
(10) Pub. No.: US 2023/0019202 A1
(43) Pub. Date: Jan. 19, 2023

(54) METHOD AND ELECTRONIC DEVICE FOR GENERATING MOLECULE SET, AND STORAGE MEDIUM THEREOF

(71) Applicant: BEIJING BAIDU NETCOM SCIENCE TECHNOLOGY CO., LTD., Beijing (CN)

(72) Inventors: Zhiyuan Chen, Beijing (CN); Xiaomin Fang, Beijing (CN); Fan Wang, Beijing (CN); Jingzhou He, Beijing (CN)

(73) Assignee: BEIJING BAIDU NETCOM SCIENCE TECHNOLOGY CO., LTD., Beijing (CN)

(21) Appl. No.: 17/936,422

(22) Filed: Sep. 29, 2022

(30) Foreign Application Priority Data
Dec. 29, 2021 (CN) 202111647796.6

(51) Int. Cl. G16C 20/50; G16C 20/70

(52) U.S. Cl. CPC G16C 20/50; G16C 20/70

(57) ABSTRACT

Embodiments of the present disclosure provide a method and electronic device for generating a molecule set and a storage medium thereof. The method obtains the first initialization molecule subset from the initialization molecule set with the pre-screening model; acquires the physical information of at least one initialization molecule in the first initialization molecule subset, and screens at least one initialization molecule based on the physical information to obtain the screened molecule set; acquires the biochemical experimental evaluation value of at least one molecule in the screened molecule set; and obtains the target molecule set based on the biochemical experimental evaluation value of at least one molecule.

### CROSS-REFERENCE TO RELATED APPLICATIONS

This application is based on and claims priority to Chinese Patent Application No. 202111647796.6, filed on Dec. 29, 2021, the entire content of which is incorporated herein by reference.

### TECHNICAL FIELD

The present disclosure relates to the technical field of computer, specifically to the technical field of de novo design of a drug, and more specifically to a method and electronic device for generating a molecule set, and a storage medium thereof.

### BACKGROUND

The goal of drug design is to find a molecule with a certain desirable property based on a huge chemical space. The de novo design of a drug, i.e. producing a new molecular entity with a desirable pharmacological property from scratch, is often considered as one of the most challenging computer-assisted tasks in the drug design, in view of the base number of the chemical space of a drug-like molecule estimated to be in the order of 10⁶⁰-10¹⁰⁰. As an important tool for the de novo design of a drug, a molecule set generation generates a new molecular structure with low-cost and high efficiency, thus accelerating the process of the drug design.

### SUMMARY

The present disclosure provides in embodiments a method and electronic device for generating a molecule set with higher efficiency, and a storage medium thereof.

According to an aspect of embodiments of the present disclosure, there is provided a method for generating a molecule set, comprising:

obtaining a first initialization molecule subset from an initialization molecule set with a pre-screening model;

acquiring physical information of at least one initialization molecule in the first initialization molecule subset, and screening said at least one initialization molecule based on the physical information, to obtain a screened molecule set;

acquiring a biochemical experimental evaluation value of at least one molecule in the screened molecule set; and

obtaining a target molecule set based on the biochemical experimental evaluation value of said at least one molecule.

In some embodiments, obtaining a first initialization molecule subset from an initialization molecule set with a pre-screening model comprises:

screening the initialization molecule set with a genetic algorithm, to obtain a second initialization molecule subset;

screening said at least one initialization molecule in the second initialization molecule subset with the pre-screening model, to obtain the first initialization molecule subset.

In some embodiments, screening said at least one initialization molecule in the second initialization molecule subset with the pre-screening model to obtain the first initialization molecule subset comprises:

acquiring a selection strategy corresponding to the pre-screening model, wherein the selection strategy comprises a molecule score and a spatial diversity condition; and

acquiring said at least one initialization molecule meeting the selection strategy from the second initialization molecule subset, to obtain the first initialization molecule subset.

In some embodiments, obtaining a target molecule set based on the biochemical experimental evaluation value of said at least one molecule comprises:

reobtaining a third initialization molecule subset and taking the third initialization molecule subset as the first initialization molecule subset, and rerunning a step of acquiring the biochemical experimental evaluation value of said at least one molecule in the screened molecule set;

stopping running a step of obtaining the third initialization molecule subset, based on a variation value of the biochemical experimental evaluation values of each molecule in the screened molecule set being less than a variation threshold.

In some embodiments, before obtaining a first initialization molecule subset from an initialization molecule set with a pre-screening model, the method further comprises:

obtaining at least one initialization seed by sampling with a neural network model;

obtaining the initialization molecule set corresponding to said at least one initialization seed with a generation model.

In some embodiments, obtaining at least one initialization seed by sampling with a neural network model comprises:

obtaining said at least one initialization seed by sampling from an initialized model latent space with the neural network model; or obtaining said at least one initialization seed by sampling from a generated space with the neural network model.

In some embodiments, after obtaining a target molecule set based on the biochemical experimental evaluation value of said at least one molecule, the method further comprises:

acquiring attribute information and verification information of at least one target molecule in the target molecule set;

training the pre-screening model based on the attribute information and the verification information of said at least one target molecule, to obtain a trained pre-screening model.

According to another aspect of embodiments of the present disclosure, there is provided an electronic device, comprising:

at least one processor; and

a memory, connected in communication with said at least one processor,

wherein the memory stores therein instructions executable by said at least one processor,

wherein said at least one processor is configured to:

obtain a first initialization molecule subset from an initialization molecule set with a pre-screening model;

acquire physical information of at least one initialization molecule in the first initialization molecule subset, and screen said at least one initialization molecule based on the physical information, to obtain a screened molecule set;

acquire a biochemical experimental evaluation value of at least one molecule in the screened molecule set; and

obtain a target molecule set based on the biochemical experimental evaluation value of said at least one molecule.

In some embodiments, said at least one processor is configured to:

screen the initialization molecule set with a genetic algorithm, to obtain a second initialization molecule subset;

screen said at least one initialization molecule in the second initialization molecule subset with the pre-screening model, to obtain the first initialization molecule subset.

In some embodiments, said at least one processor is specifically configured to:

acquire a selection strategy corresponding to the pre-screening model, wherein the selection strategy comprises a molecule score and a spatial diversity condition;

acquire said at least one initialization molecule meeting the selection strategy from the second initialization molecule subset, to obtain the first initialization molecule subset.

In some embodiments, said at least one processor is configured to:

reobtain a third initialization molecule subset and take the third initialization molecule subset as the first initialization molecule subset, and rerun a step of acquiring the biochemical experimental evaluation value of said at least one molecule in the screened molecule set;

stop running a step of obtaining the third initialization molecule subset, based on a variation value of the biochemical experimental evaluation values of each molecule in the screened molecule set being less than a variation threshold.

In some embodiments, before obtaining an initialization molecule subset from an initialization molecule set with a pre-screening model, said at least one processor is configured to:

obtain at least one initialization seed by sampling with a neural network model;

obtain the initialization molecule set corresponding to said at least one initialization seed with a generation model.

In some embodiments, said at least one processor is specifically configured to:

obtain said at least one initialization seed by sampling from an initialized model latent space with the neural network model; or

obtain said at least one initialization seed by sampling from a generated space with the neural network model.

In some embodiments, after obtaining a target molecule set based on the biochemical experimental evaluation value of said at least one molecule, said at least one processor is configured to:

acquire attribute information and verification information of at least one target molecule in the target molecule set;

train the pre-screening model based on the attribute information and the verification information of said at least one target molecule, to obtain a trained pre-screening model.

According to still another aspect of embodiments of the present disclosure, there is provided a non-transitory computer-readable storage medium having stored therein computer instructions, wherein the computer instructions cause the computer to implement a method for generating a molecule set, comprising:

obtaining a first initialization molecule subset from an initialization molecule set with a pre-screening model;

acquiring physical information of at least one initialization molecule in the first initialization molecule subset, and screening said at least one initialization molecule based on the physical information, to obtain a screened molecule set;

acquiring a biochemical experimental evaluation value of at least one molecule in the screened molecule set; and

obtaining a target molecule set based on the biochemical experimental evaluation value of said at least one molecule.

According to embodiments of the present disclosure, a first initialization molecule subset is obtained from an initialization molecule set with a pre-screening model; physical information of at least one initialization molecule in the first initialization molecule subset is acquired, and said at least one initialization molecule is screened based on the physical information to obtain a screened molecule set; a biochemical experimental evaluation value of at least one molecule in the screened molecule set is acquired; and a target molecule set is obtained based on the biochemical experimental evaluation value of said at least one molecule, thus improving the efficiency for generating the molecule set.

It should be understood that the content described herein is not intended to identify key or critical features of embodiments of the present disclosure, nor is intended to limit the scope of the present disclosure. Other features of the present disclosure will become readily appreciated from the following descriptions.

### BRIEF DESCRIPTION OF THE DRAWINGS

Drawings are explanatory, serve to explain the disclosure, and are not construed to limit embodiments of the disclosure, in which

FIG. 1 is a schematic diagram showing a method for generating a molecule set according to examples of the present disclosure;

FIG. 2 is an application scenario showing a system for achieving a method for generating a molecule set according to examples of the present disclosure;

FIG. 3 is a flow chart showing a method for generating a molecule set according to a first example of the present disclosure;

FIG. 4 is a flow chart showing a method for generating a molecule set according to a second example of the present disclosure;

FIG. 5 is an application scenario showing a step of obtaining a first initialization molecule subset according to examples of the present disclosure;

FIG. 6a is a block diagram showing a device for generating a molecule set to implement a method for generating a molecule set according to examples of the present disclosure;

FIG. 6b is a block diagram showing another device for generating a molecule set to implement a method for generating a molecule set according to examples of the present disclosure;

FIG. 6c is a block diagram showing still another device for generating a molecule set to implement a method for generating a molecule set according to examples of the present disclosure;

FIG. 6d is a block diagram showing yet another device for generating a molecule set to implement a method for generating a molecule set according to examples of the present disclosure;

FIG. 6e is a block diagram showing yet another device for generating a molecule set to implement a method for generating a molecule set according to examples of the present disclosure;

FIG. 7 is a block diagram showing an electronic device for implementing a method for generating a molecule set according to examples of the present disclosure.

### DETAILED DESCRIPTION

Illustrative embodiments of the present disclosure are described below with reference to the drawings, where various details of the embodiments of the present disclosure are included to facilitate understanding and should be considered as illustrative only. Accordingly, those of ordinary skill in the art will recognize that various changes and modifications of the embodiments described herein can be made without departing from the scope and spirit of the present disclosure. Also, descriptions of well-known functions and constructions are omitted from the following description for clarity and conciseness.

With the development of science and technology, molecule set generation is a process for automatically providing new chemical structures that meet desirable molecular properties in the best way. The molecule set generation includes ligand-based generation and target-based generation. The ligand-based generation refers to generate a new molecule based on an existing molecule that has been approved as a drug by modifying its structure with a model, which ignores target information and can only generate a molecule with a similar structure to an existing active ligand, thus presenting limited applications. By contrast, the target-based generation refers to design a molecular structure effectively binding to a target pocket according to target pocket information, which effectively combines the target information thus optimizing the target more definitely, generating an effective and highly active molecule directed to a specific target protein, and thereby presenting more realistic significance.

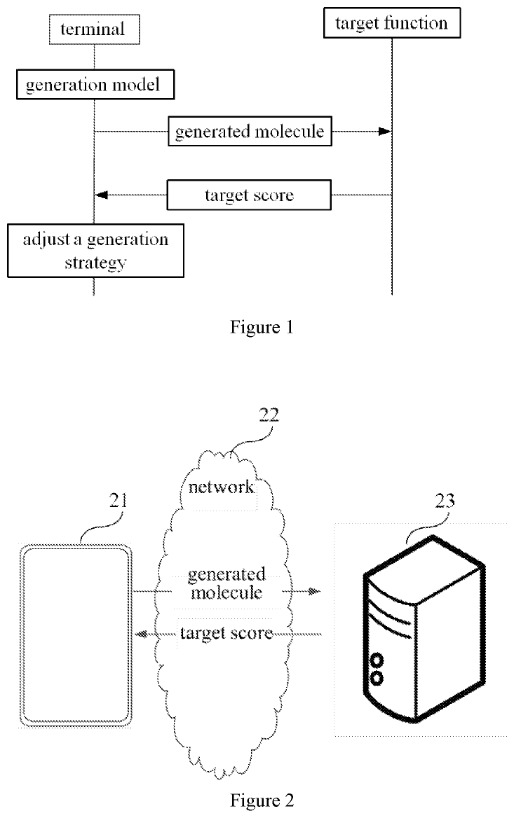

According to some examples, FIG. 1 is a schematic diagram showing a method for generating a molecule set according to examples of the present disclosure. As shown in FIG. 1, based on generating a molecule by the target-based generation, a terminal may generate the molecule with a target-based generation model. Based on obtaining the generated molecule, the terminal may call a target function to obtain a target score of the current molecule and adjust a generation strategy according to the current target score so as to maximize the generated molecule in terms of the target score.

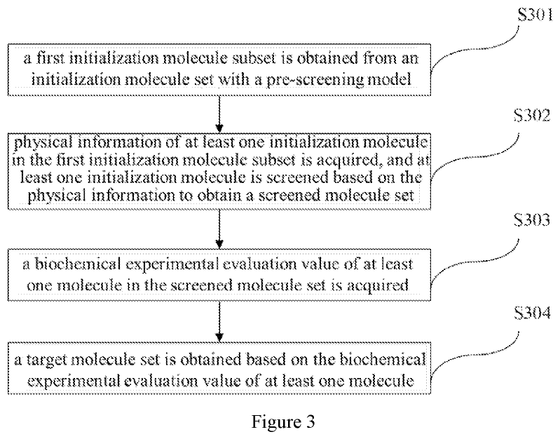

In some examples, FIG. 2 is an application scenario showing a system for achieving a method for generating a molecule set according to examples of the present disclosure. As shown in FIG. 2, a terminal 21 generates a molecule with a target-based generation model, and uploads a generated molecule to a server 23 through a network 22. Based on obtaining the generated molecule, the server 23 may call a target function to obtain a target score of a current molecule, and transmit the target score to the terminal 21 through the network 22, and then the terminal 21 may adjust a generation strategy according to the target score so as to maximize the generated molecule in terms of the target score.

It would be understood that, the target-based molecule generation requires a tremendous amount of calculations or experimental evaluations due to lack of an appropriate evaluation process designed for the generated molecule, which consumes a lot of time, computational resources, materials, etc., and is costly and of low practicality.

The present disclosure is described in detail below with reference to specific examples.

In a first example, as shown in FIG. 3 which is a flow chart showing a method for generating a molecule set according to a first example of the present disclosure, the method may be achieved via a computer program, and may be executed by a device for generating a molecule set. The computer program may be integrated into an application, or run as an independent application as a tool.

According to examples of the present disclosure, the device for generating a molecule set may be a terminal with a function of generating a molecule set. The terminal includes, but is not limited to, wearable devices, handheld devices, personal computers, tablet computers, in-vehicle devices, smart phones, computing devices, or other processing devices connected to wireless modems, etc. The terminal may be called by different names in different networks, for example: a user device, an access terminal, a user unit, a user station, a mobile station (MBS), a mobile station (MS), a remote station, a remote terminal, a mobile device, a user terminal, a terminal, a wireless communication device, a User Agent or User Apparatus, a Cellular Phone, a Cordless Phone, a Personal Digital Assistant (PDA), a 5th Generation Mobile Communication Technology (5G) network, a 4th Generation Mobile Communication Technology (4G) network, a 3rd generation mobile communication technology (3G) network or a terminal in future evolution network, etc.

Specifically, the method for generating a molecule set may include steps S301 to S304.

At the step S301, a first initialization molecule subset is obtained from an initialization molecule set with a pre-screening model.

According to some examples, the initialization molecule set refers to a set of unscreened molecules generated by a terminal. The initialization molecule set does not refer specifically to a particular fixed set. For example, based on that the number of initialization molecules changes, the initialization molecule set will change accordingly. The initialization molecule set will also change based on that the type of the initialization molecules changes.

In some examples, the first initialization molecule subset refers to a set of molecules that have the most potential for evaluation among the initialization molecule set. The first initialization molecule subset does not refer specifically to a particular fixed set. For example, based on that the initialization molecule set changes, the first initialization molecule subset will change accordingly. Based on that the pre-screening model changes, the first initialization molecule subset will also change.

In some examples, the pre-screening model refers to a model by which the terminal selects the first initialization molecule subset from the initialization molecule set. The pre-screening model does not refer specifically to a particular fixed model. Based on that the terminal obtains a model modifying instruction directed to the pre-screening model, the pre-screening model will change correspondingly. The pre-screening model includes, but is not limited to a random forest model, a graph neural network in deep learning, etc., in traditional machine learning.

It would be understood that, in the process of the terminal generating the molecule set, based on obtaining the initialization molecule set, the terminal obtains the first initialization molecule subset from the initialization molecule set with the pre-screening model.

At the step S302, physical information of at least one initialization molecule in the first initialization molecule subset is acquired, and at least one initialization molecule is screened based on the physical information to obtain a screened molecule set.

According to some examples, the physical information refers to information that a molecule exhibits without undergoing chemical changes. The physical information does not refer specifically to particular fixed information. The physical information includes, but is not limited to binding free energy, binding activity, toxicity, free energy perturbation, etc.

In some examples, the binding free energy refers to interaction between a ligand and a receptor. The higher its negative value is, the stronger the bonding is and the greater the energy required for breaking a bond is. If the binding free energy is a positive value, it is impossible to form surface bonding spontaneously. The free energy perturbation is a common approach used to calculate the free energy.

In some examples, the term “screening” used herein refers to a process by which the terminal performs a cascade of filters on at least one initialization molecule to obtain the molecules that have the most potential for evaluation. The means for screening does not refer specifically to a particular fixed means. Based on that the terminal obtains a means modifying instruction directed to the means for screening, the means for screening will change correspondingly. The means for screening may be, for example, screening by means of a computational model, etc.

In some examples, all of screened molecules that have the potential for evaluation are put into a same set, thereby obtaining the screened molecule set. The screened molecule set does not refer specifically to a particular fixed set. For example, based on that the means for screening changes, the screened molecule set will change accordingly. Based on that the first initialization molecule subset changes, the screened molecule set will also change.

It would be understood that, based on obtaining the first initialization molecule subset, the terminal acquires the physical information of at least one initialization molecule in the first initialization molecule subset. Based on acquiring the physical information of at least one initialization molecule, the terminal screens at least one initialization molecule based on the physical information so as to obtain the screened molecule set.

At the step S303, a biochemical experimental evaluation value of at least one molecule in the screened molecule set is acquired.

According to some examples, the biochemical experimental evaluation value refers to an actual attribute value of the molecule that is determined through experiments. The attribute value includes, but is not limited to binding free energy, binding activity, toxicity, free energy perturbation, etc. The biochemical experimental evaluation value does not refer specifically to a particular fixed evaluation value. For example, based on that the type of the attribute value changes, the biochemical experimental evaluation value will change accordingly. Based on that the molecule changes, the biochemical experimental evaluation value to which the molecule corresponds will also change.

It would be understood that, based on obtaining the screened molecule set, the terminal acquires the biochemical experimental evaluation value of at least one molecule in the screened molecule set.

At the step S304, a target molecule set is obtained based on the biochemical experimental evaluation value of at least one molecule.

According to some examples, the target molecule set refers to a set of molecules of which its molecule-generating quality reaches a target molecule-generating quality obtained from the screened molecule set by the terminal, based on the biochemical experimental evaluation value of at least one molecule. The target molecule set does not refer specifically to a particular fixed set. For example, based on that the screened molecule set changes, the target molecule set will change accordingly. Based on the target molecule-generating quality changes, the target molecule set will also change.

It would be understood that, based on acquiring the biochemical experimental evaluation value of at least one molecule, the terminal obtains the target molecule set of which its molecule-generating quality reaches the target molecule-generating quality.

According to examples of the present disclosure, the method for generating a molecule set obtains the first initialization molecule subset from the initialization molecule set with the pre-screening model; acquires the physical information of at least one initialization molecule in the first initialization molecule subset, and screens at least one initialization molecule based on the physical information to obtain the screened molecule set; acquires the biochemical experimental evaluation value of at least one molecule in the screened molecule set; and obtains the target molecule set based on the biochemical experimental evaluation value of at least one molecule. Therefore, the method calculates the physical information and the biochemical experimental evaluation value to obtain the target molecule set, decreasing the number of calculations and improving the efficiency of the generation of the molecule set, and thus reducing the consumption of resource costs and improving the practicality, thereby improving the user experience.

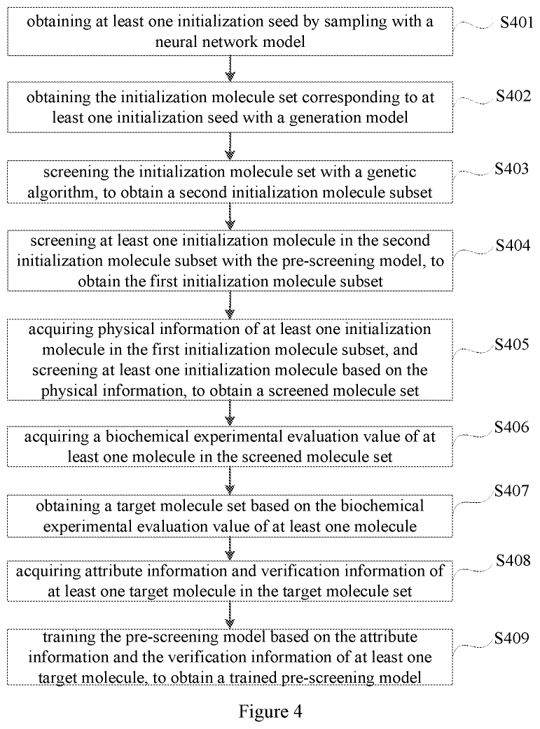

FIG. 4 is a flow chart showing a method for generating a molecule set according to a second example of the present disclosure, including steps S401 to S409.

Specifically, at the step S401, at least one initialization seed is obtained by sampling with a neural network model.

According to some examples, the neural network model refers to a mathematical model described on the basis of a mathematical model of neurons and characterized by a network topology, node characteristics and learning rules. The neural network model does not refer specifically to a particular fixed model. The neural network model includes, but is not limited to, a Back propagation (BP) neural network model, a Hopfield neural network model, an Adaptive Resonance Theory (ART) neural network model, a Kohonen network model, etc.

In some examples, the initialization seed refers to a seed by which the terminal generates the initialization molecule. The initialization seed does not refer specifically to a particular fixed seed. For example, based on that the neural network model changes, the initialization seed will change. The initialization seed will also change based on that the means of sampling changes.

In some examples, the terminal obtains the initialization seed by sampling from a model latent space, or from a generated space, with the neural network model, where by means of sampling from the model latent space, the terminal obtains at least one initialization seed by sampling from an initialized model latent space with the neural network model; by means of sampling from the generated space, the terminal obtains at least one initialization seed by sampling from the generated space with the neural network model, and thus improving the accuracy for the terminal to obtain the initialization seed.

In some examples, the model latent space refers to a data space after original data is compressed by the neural network model. The model latent space does not refer specifically to a particular fixed space. For example, based on that the original data changes, the model latent space will change as well. Based on that the neural network model changes, the model latent space will also change. For improving the accuracy of sampling, the model latent space may be, for example, a standard normal distribution.

It would be understood that, in the process of generating the molecule set, the terminal obtains at least one initialization seed by sampling with the neural network model.

At the step S402, the initialization molecule set corresponding to at least one initialization seed is obtained with a generation model.

The specific process is described above and will not be repeated here.

According to some examples, the generation model refers to a model with which the terminal obtains the initialization molecule set corresponding to said at least one initialization seed. The generation model does not specifically refer to a particular fixed model. The generation model includes, but is not limited to, Generative Adversarial Network (GAN), Variational Autoencoder (VAE), Flow, etc.

It would be understood that, based on obtaining at least one initialization seed, via the generation model, the terminal obtains the initialization molecule set corresponding to at least one initialization seed. The generation model does not specifically refer to a particular fixed model, thus reducing the dependence of the method for generating a molecule set on the model, thereby improving the flexibility for executing the method for generating a molecule set.

At the step S403, the initialization molecule set is screened with a genetic algorithm to obtain a second initialization molecule subset.

According to some examples, the genetic algorithm (GA) refers to a calculation model that simulates the natural selection of Darwinian biological evolution and the biological evolutionary process with genetics mechanism, and is a method to search for the optimal solution by simulating the natural evolutionary process. The genetic algorithm may convert a problem solving process into a process similar to the crossover, mutation or the like of a chromosomal gene in biological evolution by a computer simulation in a mathematical way.

In some examples, the second initialization molecule subset refers to a set of roughly screened molecules obtained by screening the initialization molecule set by the terminal with the genetic algorithm. The second initialization molecule subset does not refer specifically to a particular fixed set. For example, based on that the initialization molecule set changes, the second initialization molecule subset will also change. Based on that the genetic algorithm changes, the second initialization molecule subset will also change.

It would be understood that, based on obtaining the initialization molecule set, the terminal screens the initialization molecule set with the genetic algorithm thereby obtaining the second initialization molecule subset.

At the step S404, at least one initialization molecule in the second initialization molecule subset is screened with the pre-screening model, to obtain the first initialization molecule subset.

The specific process is described above and will not be repeated here.

According to some examples, in the process of screening at least one initialization molecule in the second initialization molecule subset with the pre-screening model, the terminal may acquire a selection strategy corresponding to the pre-screening model. Based on obtaining the selection strategy, the terminal may acquire at least one initialization molecule that meets the selection strategy from the second initialization molecule subset, thereby obtaining the first initialization molecule subset, which improves the accuracy for the terminal to obtain the first initialization molecule subset.

In some examples, the selection strategy refers to a point selection principle employed for screening at least one initialization molecule in the second initialization molecule subset with the pre-screening model. The selection strategy does not refer specifically to a particular fixed strategy. Types of the selection strategy include, but are not limited to, active learning, Bayesian optimization, Constrained Global Optimization, determinant point process (DPP), etc.

In some examples, the content of the selection strategy includes, but is not limited to, a molecule score, a spatial diversity condition, etc. Accordingly, in the process of obtaining the first initialization molecule subset, the terminal may take into account of both the molecule score and the spatial diversity condition, making the screened molecules meet a high molecule score while have a high degree of dispersion in the spatial distribution, thus improving the diversity and novelty of the molecules in the first initialization molecule subset.

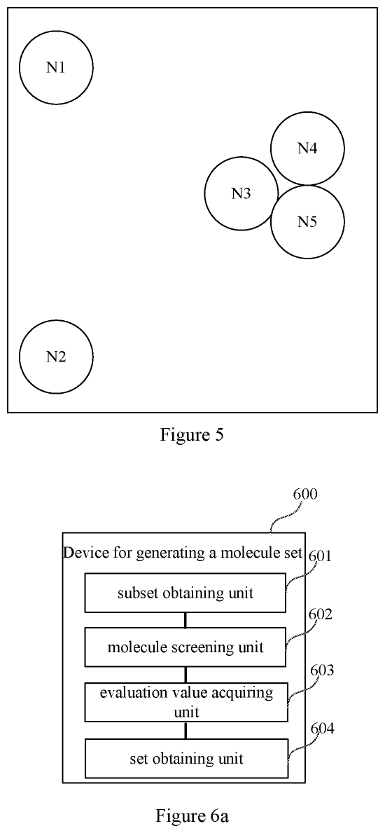

For example, in the process of obtaining the first initialization molecule subset, the terminal obtains five molecules with similar molecule scores from the second initialization molecule subset, three of which have similar positions. Thus, the terminal may select the molecule with the highest molecule score among the three molecules having similar positions, and the remaining two molecules that are not closely positioned, into the first initialization molecule subset. As shown in FIG. 5, the terminal obtains five molecules with similar molecule scores from the second initialization molecule subset, i.e., N1-N5, where the molecule scores of N3, N4, and N5 are 90 for N3, 85 for N4, and 80 for N5, and thus the terminal puts the three molecules N1, N2, and N3 into the first initialization molecule subset.

It would be understood that, based on obtaining the second initialization molecule subset, the terminal screens at least one initialization molecule in the second initialization molecule subset with the pre-screening model, thereby obtaining the first initialization molecule subset.

At the step S405, physical information of at least one initialization molecule in the first initialization molecule subset is acquired, and at least one initialization molecule is screened based on the physical information, to obtain a screened molecule set.

The specific process is described above and will not be repeated here.

According to some examples, in the process of the terminal screening at least one initialization molecule based on the physical information, aspects of screening calculation include, but are not limited to the calculations of binding free energy, binding activity, toxicity, free energy perturbation, etc., where the terminal may calculate the binding free energy by molecule docking, calculate the binding activity by a molecule activity prediction model, calculate the toxicity by a toxicity (Admet) prediction model, and calculate the free energy perturbation by delta delta G. Thus, the terminal may be effectively and flexibly combined with various techniques such as docking, the Admet prediction model, the molecule activity prediction models, etc., independent of the form of the generation model to be combined.

For example, based on the physical information of any one of the initialization molecules, the terminal performs the screening calculation on the initialization molecule and determines that the initialization molecule has a binding free energy of A, a binding activity of B, a toxicity of C, and a free energy perturbation of D, where the binding free energy A is less than a binding free energy threshold, the binding activity B is greater than a binding activity threshold, the toxicity C is greater than a toxicity threshold, and the free energy perturbation D is greater than a free energy perturbation threshold, and then this initialization molecule may be put into the screened molecule set by the terminal. It would be understood that, based on obtaining the first initialization molecule subset, the terminal acquires the physical information of at least one initialization molecule in the first initialization molecule subset. Based on acquiring the physical information of at least one initialization molecule, the terminal screens at least one initialization molecule based on the physical information, thereby obtaining the screened molecule set.

At the step S406, a biochemical experimental evaluation value of at least one molecule in the screened molecule set is acquired.

The specific process is described above and will not be repeated here.

According to some examples, based on at least one attribute value of any one of the molecules in the screened molecule set acquired by biochemical experiments, the terminal may determine an average or weighted average of the at least one attribute value to obtain the biochemical experimental evaluation value of the molecule. For example, based on that the attribute value 1 of molecule M in the screened molecule set is M1, the attribute value 2 is M2, and the attribute value 3 is M3, which are acquired through the biochemical experiments, the terminal may acquire the biochemical experimental evaluation value of the molecule as (M1+M2+M3)/3.

It would be understood that, based on obtaining the screened molecule set, the terminal acquires the biochemical experimental evaluation value of at least one molecule in the screened molecule set.

At the step S407, a target molecule set is obtained based on the biochemical experimental evaluation value of at least one molecule.

The specific process is described above and will not be repeated here.

According to some examples, with an iterative way, the terminal iterates the steps S401-S406 at least once based on the biochemical experimental evaluation value of at least one molecule, until the acquired molecule is of a molecule-generating quality reaching the target molecule-generating quality, thereby obtaining the target molecule set.

In some examples, during the iteration, the terminal reobtains a third initialization molecule subset and takes the third initialization molecule subset as the first initialization molecule subset, and reruns the step of acquiring the biochemical experimental evaluation value of at least one molecule in the screened molecule set; and stops running the step of obtaining the third initialization molecule subset, based on a variation value of the biochemical experimental evaluation values of each molecule in the screened molecule set being less than a variation threshold, and thus improving the quality of the acquired target molecule set.

In some examples, the third initialization molecule subset refers to a set of molecules having the most potential for evaluation and reobtained by the terminal according to the steps S401-S404. The third initialization molecule subset does not refer specifically to a particular fixed set. For example, based on that the initialization molecule set changes, the third initialization molecule subset will also change. Based on that the pre-screening model changes, the third initialization molecule subset will also change.

In some examples, the variation threshold does not refer specifically to a particular fixed threshold. Based on that the terminal obtains a threshold modifying instruction directed to the variation threshold, the variation threshold will change correspondingly.

It would be understood that, based on obtaining the biochemical experimental evaluation value of at least one molecule, the terminal obtains the target molecule set of which its molecule-generating quality reaching the target molecule-generating quality.

At the step S408, attribute information and verification information of at least one target molecule in the target molecule set are acquired.

According to some examples, the attribute information refers to the physical information and biochemical experimental information of the target molecule. The attribute information does not refer specifically to particular fixed information. The attribute information includes, but is not limited to binding free energy, binding activity, toxicity, free energy perturbation, etc.

In some examples, the verification information refers to at least one attribute value in the biochemical experimental evaluation value of the target molecule. The verification information includes, but is not limited to binding free energy, binding activity, toxicity, free energy perturbation, etc. It would be understood that, based on obtaining the target molecule set, the terminal acquires the attribute information and the verification information of at least one target molecule in the target molecule set.

At the step S409, the pre-screening model is trained based on the attribute information and the verification information of at least one target molecule, to obtain a trained pre-screening model.

It would be understood that, based on acquiring the attribute information and the verification information of at least one target molecule in the target molecule set, the terminal takes the target molecule and the attribute information and the verification information thereof as a training sample to train the pre-screening model, thereby obtaining the trained pre-screening model.

According to examples of the present disclosure, the terminal obtains at least one initialization seed by sampling with the neural network model, and obtains the initialization molecule set corresponding to at least one initialization seed with the generation model, thus improving the efficiency of obtaining the initialization molecule set. Further, the initialization molecule set is screened with the genetic algorithm to obtain the second initialization molecule subset, and at least one initialization molecule in the second initialization molecule subset is screened with the pre-screening model, to obtain the first initialization molecule subset, thus improving the accuracy of obtaining the first initialization molecule subset. Besides, the physical information of at least one initialization molecule in the first initialization molecule subset is acquired, and at least one initialization molecule is screened based on the physical information to obtain the screened molecule set; the biochemical experimental evaluation value of at least one molecule in the screened molecule set is acquired; and the target molecule set is obtained based on the biochemical experimental evaluation value of at least one molecule. Therefore, the method calculates the physical information and the biochemical experimental evaluation value to obtain the target molecule set, decreasing the number of calculations and improving the efficiency of the generation of the molecule set, and thus reducing the consumption of resource costs and improving the practicality, thereby improving the user experience. Furthermore, the attribute information and the verification information of at least one target molecule in the target molecule set are acquired, and the pre-screening model is trained based on the attribute information and the verification information of at least one target molecule, to obtain the trained pre-screening model, thus improving the accuracy of the pre-screening model.

The collection, storage, use, processing, transmission, provision and disclosure of the user's personal information involved in the embodiments of the present disclosure are in compliance with relevant laws and regulations, and do not violate public order and moral.

Examples related to devices in the present disclosure are provided below, which may be used for implementing the methods according to examples of the present disclosure. For details not disclosed in the examples related to the devices, please refer to the examples of the methods in the present disclosure.

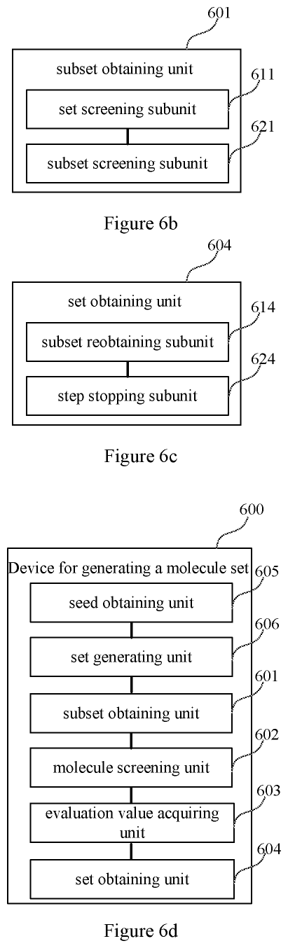

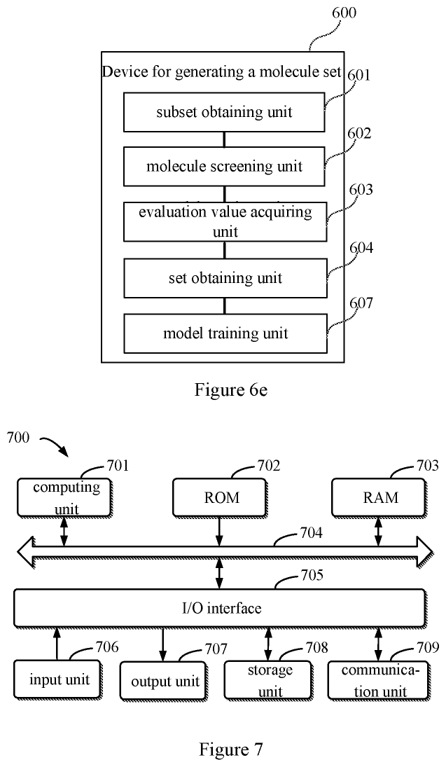

FIG. 6a is a block diagram showing a device for generating a molecule set to implement a method for generating a molecule set according to examples of the present disclosure. The device 600 for generating a molecule set may be as an entire or part of a device through software, hardware or a combination thereof. As shown in FIG. 6a, the device 600 for generating a molecule set includes a subset obtaining unit 601, a molecule screening unit 602, an evaluation value acquiring unit 603 and a set obtaining unit 604.

The subset obtaining unit 601 is configured to obtain a first initialization molecule subset from an initialization molecule set with a pre-screening model.

The molecule screening unit 602 is configured to acquire physical information of at least one initialization molecule in the first initialization molecule subset, and screen at least one initialization molecule based on the physical information, to obtain a screened molecule set.

The evaluation value acquiring unit 603 is configured to acquire a biochemical experimental evaluation value of at least one molecule in the screened molecule set.

The set obtaining unit 604 is configured to obtain a target molecule set based on the biochemical experimental evaluation value of at least one molecule.

In some examples, FIG. 6b is a block diagram showing another device for generating a molecule set to implement a method for generating a molecule set according to examples of the present disclosure. As shown in FIG. 6b, the subset obtaining unit 601 includes a set screening subunit 611 and a subset screening subunit 621. When the subset obtaining unit 601 is configured to obtain a first initialization molecule subset from an initialization molecule set with a pre-screening model,

the set screening subunit 611 is configured to screen the initialization molecule set with a genetic algorithm, to obtain a second initialization molecule subset;

the subset screening subunit 621 is configured to screen at least one initialization molecule in the second initialization molecule subset with the pre-screening model, to obtain the first initialization molecule subset.

In some examples, when the subset screening subunit 621 is configured to screen at least one initialization molecule in the second initialization molecule subset with the pre-screening model to obtain the first initialization molecule subset, the subset screening subunit 621 is specifically configured to:

acquire a selection strategy corresponding to the pre-screening model, where the selection strategy includes a molecule score and a spatial diversity condition;

acquire at least one initialization molecule meeting the selection strategy from the second initialization molecule subset, to obtain the first initialization molecule subset.

In some examples, FIG. 6c is a block diagram showing still another device for generating a molecule set to implement a method for generating a molecule set according to examples of the present disclosure. As shown in FIG. 6c, the set obtaining unit 604 includes a subset reobtaining subunit 614 and a step stopping subunit 624, when the set obtaining unit 604 is configured to obtain a target molecule set based on the biochemical experimental evaluation value of at least one molecule,

the subset reobtaining subunit 614 is configured to reobtain a third initialization molecule subset and take the third initialization molecule subset as the first initialization molecule subset, and rerun a step of acquiring the biochemical experimental evaluation value of at least one molecule in the screened molecule set;

the step stopping subunit 624 is configured to stop running a step of obtaining the third initialization molecule subset, based on a variation value of the biochemical experimental evaluation values of each molecule in the screened molecule set being less than a variation threshold.

In some examples, FIG. 6d is a block diagram showing yet another device for generating a molecule set to implement a method for generating a molecule set according to examples of the present disclosure. As shown in FIG. 6d, the device 600 for generating a molecule set further includes a seed obtaining unit 605 and a set generating unit 606, before obtaining a first initialization molecule subset from an initialization molecule set with a pre-screening model,

the seed obtaining unit 605 is configured to obtain at least one initialization seed by sampling with a neural network model;

the set generating unit 606 is configured to obtain the initialization molecule set corresponding to at least one initialization seed with a generation model.

In some examples, when the seed obtaining unit 605 is configured to obtain at least one initialization seed by sampling with a neural network model, the seed obtaining unit 605 is specifically configured to:

obtain at least one initialization seed by sampling from an initialized model latent space with the neural network model; or

obtain at least one initialization seed by sampling from a generated space with the neural network model.

In some examples, FIG. 6e is a block diagram showing yet another device for generating a molecule set to implement a method for generating a molecule set according to examples of the present disclosure. As shown in FIG. 6e, the device 600 for generating a molecule set further includes a model training unit 607, after obtaining a target molecule set based on the biochemical experimental evaluation value of at least one molecule, the model training unit 607 is configured to:

acquire attribute information and verification information of at least one target molecule in the target molecule set; train the pre-screening model based on the attribute information and the verification information of at least one target molecule, to obtain a trained pre-screening model.

It should be noted that, the devices for generating a molecule set provided in the above examples are only illustrated with division regarding each functional module as an example, when performing the method for generating a molecule set. In practice, the above-mentioned functions may be assigned to different functional modules according to the needs, i.e., the internal structure of the device is divided into different functional modules to perform all or part of the above-described functions. In addition, the device for generating a molecule set provided in the above examples belongs to the same concept as the method for generating a molecule set according to examples of the present disclosure, and its implementation process is detailed in the method embodiment, which will not be repeated here.

The above-mentioned serial numbers of the examples of the present disclosure are for descriptive purposes only and do not represent the advantages or disadvantages of the examples.

According to one or more examples of the present disclosure, the subset obtaining unit obtains the first initialization molecule subset from the initialization molecule set with the pre-screening model; the molecule screening unit acquires the physical information of at least one initialization molecule in the first initialization molecule subset, and screens at least one initialization molecule based on the physical information to obtain the screened molecule set; the evaluation value acquiring unit acquires the biochemical experimental evaluation value of at least one molecule in the screened molecule set; and the set obtaining unit obtains the target molecule set based on the biochemical experimental evaluation value of at least one molecule. Therefore, the device calculates the physical information and the biochemical experimental evaluation value to obtain the target molecule set, decreasing the number of calculations and improving the efficiency of the generation of the molecule set, and thus reducing the consumption of resource costs and improving the practicality, thereby improving the user experience.

The collection, storage and use of the user's personal information involved in the embodiments of the present disclosure are in compliance with relevant laws and regulations, and do not violate public order and moral.

According to examples of the present disclosure, there are further provided an electronic device, a computer-readable storage medium and a computer program product.

FIG. 7 is a block diagram showing an electronic device 700 according to examples of the present disclosure. The electronic devices are intended to represent various forms of digital computers, such as a laptop, a desktop, a workstation, a personal digital assistant, a server, a blade server, a mainframe computer, and other suitable computers. The electronic devices may also represent various forms of mobile devices, such as a personal digital processor, a cellular phone, a smart phone, a wearable device, and other similar computing devices. The components shown herein, their connections and relationships, and their functions are described as examples only, and are not intended to limit implementations of the present disclosure described and/or claimed herein.

As shown in FIG. 7, the device 700 includes a computing unit 701 to perform various appropriate actions and processes according to a computer program instruction stored in a read only memory (ROM) 702 or a computer program loaded from a storage unit 708 into a random access memory (RAM) 703. The RAM 703 may also stores therein various programs and data required for the operation of the storage device 700. The computing unit 701, the ROM 702, and the RAM 703 are connected to each other through a bus 704. An input/output (I/O) interface 605 is also connected to the bus 704.

Components in the device 700, connected to the I/O interface 705, includes: an input unit 706, such as a keyboard and a mouse; an output unit 707, such as various types of displays and speakers; a storage unit 708, such as a disk and an optical disk; and a communication unit 709, such as a network card, a modem and a wireless communication transceiver. The communication unit 709 allows the device 700 to exchange information/data with other devices through a computer network such as the Internet and/or various telecommunication networks.

The computing unit 701 may be various general-purpose and/or special-purpose processing components with processing and computing capabilities.

Some examples of the computing unit 701 include, but are not limited to, a central processing unit (CPU), a graphic processing unit (GPU), various specialized artificial intelligence (AI) computing chips, various computing units that run machine learning model algorithms, a digital signal processor (DSP), and any suitable processor, controller, microcontroller, etc. The computing unit 701 performs various methods and processes described above, such as a method for generating a molecule set. For example, in some embodiments, the method for generating a molecule set may be implemented as a computer software program that is tangibly embodied on a machine-readable medium, such as the storage unit 708. In some embodiments, a part or all of the computer program may be loaded and/or installed on the device 700 via the ROM 702 and/or the communication unit 709. When a computer program is loaded into the RAM 703 and executed by the computing unit 701, one or more steps of the method for generating a molecule set described above may be performed. Alternatively, in other embodiments, the computing unit 701 may be configured to perform the method for generating a molecule set in any other suitable manner (e.g., by means of firmware).

Various implementations of the system and technique described herein above may be implemented in a digital electronic circuit, an integrated circuit system, a field programmable gate array (FPGA), an application specific integrated circuit (ASIC), an application specific standard product (ASSP), a system-on-chip (SOC), a complex programmable logic device (CPLD), a computer hardware, a firmware, a software, and/or a combination thereof. These various embodiments may include implementation in one or more computer programs executable and/or interpretable on a programmable system including at least one programmable processor. The programmable processor may be a special-purpose or general-purpose programmable processor, which may receive data and instructions from a storage system, at least one input device and at least one output device, and may transmit data and instructions to the storage system, the at least one input device, and the at least one output device.

A program code for implementing the methods of the present disclosure may be written in any combination of one or more programming languages. The program code may be provided to a processor or controller of a general computer, a dedicated computer, or other programmable data processing devices, such that the program code, when executed by the process or or controller, causes the functions and/or operations specified in the flow chart and/or the block diagram are (is) performed. The program code can be executed entirely on the machine, partly on the machine, as a stand-alone software package partly on a machine and partly on a remote machine or entirely on a remote machine or server.

In the context of the present disclosure, a machine-readable medium may be a tangible medium that may contain or store a program for use by or in connection with an instruction execution system, apparatus, or device. The machine-readable medium may be a machine-readable signal medium or a machine-readable storage medium. A machine-readable medium may include, but is not limited to, an electronic, magnetic, optical, electromagnetic, infrared, or semiconductor system, apparatus, or device, or any suitable combination of the foregoing. More specific examples of the machine-readable storage medium include an electrical connection based on one or more wires, a portable computer disk, a hard disk, a random access memory (RAM), a read-only memory (ROM), an erasable programmable read-only memory (EPROM or flash memory), a fiber optics, compact disc read-only memory (CD-ROM), an optical storage device, a magnetic storage device, or any suitable combination of the foregoing.

To provide interaction with a user, the system and technique described herein may be implemented on a computer having a display device (e.g., a cathode ray tube (CRT) or a liquid crystal display (LCD)) for displaying information for the user; and a keyboard and a pointing device (e.g., a mouse or a trackball) through which a user can provide an input to the computer. Other types of devices can also be used to provide interaction with the user, for example, the feedback provided to the user may be any form of sensory feedback (e.g., visual feedback, auditory feedback, or tactile feedback); and may be in any form (including acoustic input, voice input, or tactile input) to receive the input from the user.

The system and technique described herein may be implemented on a computing system that includes a back-end component (e.g., a data server), a computing system that includes a middleware component (e.g., an application server), a computing system that includes a front-end component (e.g., a user computer with a graphical user interface or a web browser through which a user can interact with implementations of the system and technique described herein), a computing system that includes said backend component, said middleware component, and said front-end components or any combination thereof. The components of the system may be interconnected by any form or medium of digital data communication (e.g., a communication network). Examples of the communication network include: a Local Area Network (LAN), a Wide Area Network (WAN), an Internet and a blockchain network.

The computer system may include a client and a server. The client and server are generally remote from each other and usually interact through a communication network. A relationship between the client and the server is generated by a computer program running on a corresponding computer and having a client-server relationship with each other. The server may be a cloud server, also known as a cloud computing server or a cloud host, which is a host product in the cloud computing service system, and solves the defects of difficult management and weak business expansion in a traditional physical host and a virtual private server (“VPS” for short). The server may also be a server of a distributed system, or a server combined with a blockchain.

It should be understood that the steps may be reordered, added or deleted by using the various forms of flows shown above. For example, the steps described in the present disclosure may be executed in parallel, sequentially, or in different orders, as long as the desired results of the technical solutions disclosed in the present disclosure can be achieved, and no limitation is imposed herein.

The above-mentioned specific embodiments do not limit the protection scope of the present disclosure. It should be understood by those skilled in the art that various modifications, combinations, sub-combinations and replacements may be made depending on design requirements and other factors. Any modifications, equivalent replacements, and improvements made within the spirit and principles of the present disclosure should be included within the protection scope of the present disclosure.

What is claimed is:

1. A method for generating a molecule set, comprising:
obtaining a first initialization molecule subset from an initialization molecule set with a pre-screening model;
acquiring physical information of at least one initialization molecule in the first initialization molecule subset, and screening said at least one initialization molecule based on the physical information, to obtain a screened molecule set;
acquiring a biochemical experimental evaluation value of at least one molecule in the screened molecule set; and
obtaining a target molecule set based on the biochemical experimental evaluation value of said at least one molecule.

2. The method according to claim 1, wherein obtaining a first initialization molecule subset from an initialization molecule set with a pre-screening model comprises:
screening the initialization molecule set with a genetic algorithm, to obtain a second initialization molecule subset;
screening said at least one initialization molecule in the second initialization molecule subset with the pre-screening model, to obtain the first initialization molecule subset.

3. The method according to claim 2, wherein screening said at least one initialization molecule in the second initialization molecule subset with the pre-screening model to obtain the first initialization molecule subset comprises:
acquiring a selection strategy corresponding to the pre-screening model, wherein the selection strategy comprises a molecule score and a spatial diversity condition; and
acquiring said at least one initialization molecule meeting the selection strategy from the second initialization molecule subset, to obtain the first initialization molecule subset.

4. The method according to claim 1, wherein obtaining a target molecule set based on the biochemical experimental evaluation value of said at least one molecule comprises:
reobtaining a third initialization molecule subset and taking the third initialization molecule subset as the first initialization molecule subset, and rerunning a step of acquiring the biochemical experimental evaluation value of said at least one molecule in the screened molecule set;
stopping running a step of obtaining the third initialization molecule subset, based on a variation value of the biochemical experimental evaluation values of each molecule in the screened molecule set being less than a variation threshold.

5. The method according to claim 1, wherein before obtaining a first initialization molecule subset from an initialization molecule set with a pre-screening model, the method further comprises:
obtaining at least one initialization seed by sampling with a neural network model;
obtaining the initialization molecule set corresponding to said at least one initialization seed with a generation model.

6. The method according to claim 5, wherein obtaining at least one initialization seed by sampling with a neural network model comprises:
obtaining said at least one initialization seed by sampling from an initialized model latent space with the neural network model; or
obtaining said at least one initialization seed by sampling from a generated space with the neural network model.

7. The method according to claim 1, wherein after obtaining a target molecule set based on the biochemical experimental evaluation value of said at least one molecule, the method further comprises:
acquiring attribute information and verification information of at least one target molecule in the target molecule set;
training the pre-screening model based on the attribute information and the verification information of said at least one target molecule, to obtain a trained pre-screening model.

8. An electronic device, comprising:
at least one processor; and
a memory, connected in communication with said at least one processor,
wherein the memory stores therein instructions executable by said at least one processor,
wherein said at least one processor is configured to:
obtain a first initialization molecule subset from an initialization molecule set with a pre-screening model;
acquire physical information of at least one initialization molecule in the first initialization molecule subset, and screen said at least one initialization molecule based on the physical information, to obtain a screened molecule set;
acquire a biochemical experimental evaluation value of at least one molecule in the screened molecule set; and
obtain a target molecule set based on the biochemical experimental evaluation value of said at least one molecule.

9. The electronic device according to claim 8, wherein said at least one processor is configured to:
screen the initialization molecule set with a genetic algorithm, to obtain a second initialization molecule subset;
screen said at least one initialization molecule in the second initialization molecule subset with the pre-screening model, to obtain the first initialization molecule subset.

10. The electronic device according to claim 9, wherein said at least one processor is specifically configured to:
acquire a selection strategy corresponding to the pre-screening model, wherein the selection strategy comprises a molecule score and a spatial diversity condition;
acquire said at least one initialization molecule meeting the selection strategy from the second initialization molecule subset, to obtain the first initialization molecule subset.

11. The electronic device according to claim 8, wherein said at least one processor is configured to:
reobtain a third initialization molecule subset and take the third initialization molecule subset as the first initialization molecule subset, and rerun a step of acquiring the biochemical experimental evaluation value of said at least one molecule in the screened molecule set;
stop running a step of obtaining the third initialization molecule subset, based on a variation value of the biochemical experimental evaluation values of each molecule in the screened molecule set being less than a variation threshold.

12. The electronic device according to claim 8, wherein, before obtaining an initialization molecule subset from an initialization molecule set with a pre-screening model, said at least one processor is configured to:
obtain at least one initialization seed by sampling with a neural network model;
obtain the initialization molecule set corresponding to said at least one initialization seed with a generation model.

13. The electronic device according to claim 12, wherein said at least one processor is specifically configured to:
obtain said at least one initialization seed by sampling from an initialized model latent space with the neural network model; or
obtain said at least one initialization seed by sampling from a generated space with the neural network model.

14. The electronic device according to claim 8, wherein, after obtaining a target molecule set based on the biochemical experimental evaluation value of said at least one molecule, said at least one processor is configured to:
acquire attribute information and verification information of at least one target molecule in the target molecule set;
train the pre-screening model based on the attribute information and the verification information of said at least one target molecule, to obtain a trained pre-screening model.

15. A non-transitory computer-readable storage medium having stored therein computer instructions, wherein the computer instructions cause the computer to implement a method for generating a molecule set, comprising:
obtaining a first initialization molecule subset from an initialization molecule set with a pre-screening model;
acquiring physical information of at least one initialization molecule in the first initialization molecule subset, and screening said at least one initialization molecule based on the physical information, to obtain a screened molecule set;
acquiring a biochemical experimental evaluation value of at least one molecule in the screened molecule set; and
obtaining a target molecule set based on the biochemical experimental evaluation value of said at least one molecule.
